(19) United States
(12) Patent Application Publication
(10) Pub. No.: US 2023/0392115 A1
(43) Pub. Date: Dec. 7, 2023

(54) METHODS FOR MAKING AND USING DIFFERENTIATED NEURAL CELLS

(71) Applicant: Research Development Foundation, Carson City, NV (US)

(72) Inventors: Olivier PREYNAT-SEAUVE, Carson City, NV (US); Karl-Heinz KRAUSE, Carson City, NV (US)

(73) Assignee: Research Development Foundation, Carson City, NV (US)

(21) Appl. No.: 18/248,739

(22) PCT Filed: Oct. 12, 2021
(86) PCT No.: PCT/US2021/054462

§ 371 Date: Apr. 12, 2023

Related U.S. Application Data
(60) Provisional application No. 63/090,590, filed on Oct. 12, 2020.

(51) Int. Cl. C12N 5/0793; A61P 25/28

(52) U.S. Cl. CPC C12N 5/0619; A61P 25/28; C12N 2501/999; C12N 2500/99; C12N 2533/52; C12N 2506/03

(57) ABSTRACT

The current disclosure provides for methods for differentiating stem and progenitor cells into neural cells through an approach that excludes the use of SMAD or Noggin inhibition. Aspects of the disclosure relate to a method for differentiating stem or progenitor cells into neural cells, the method comprising contacting the cells with a compound selected from DMH1, DMH2, K02288, A8301, or combinations thereof.

### CROSS REFERENCE TO RELATED APPLICATIONS

This application claims the benefit of priority to U.S. Provisional Patent Application Ser. No. 63/090,590, filed Oct. 12, 2020, hereby incorporated by reference in its entirety.

The instant application contains a Sequence Listing which has been submitted in ASCII format and is hereby incorporated by reference in its entirety. Said ASCII copy, created on Oct. 8, 2021, is named CLFRPO403WO_ST25.txt and is 1,436 bytes in size.

### BACKGROUND OF THE INVENTION

### I. Field of the Invention

This invention relates to the field of cell biology and treatment of disease.

### II. Background

Cell populations that retain the ability to differentiate into numerous specialized cell types are useful for developing large numbers of lineage specific differentiated cell populations. These lineage specific differentiated cell populations are contemplated to find use in cell replacement therapies for patients with diseases resulting in loss of function of a defined cell population. In addition to their direct therapeutic value, lineage specific differentiated cells are also valuable research tools for a variety of purposes including in vitro screening assays to identify, confirm, and test for specification of function or for testing delivery of therapeutic molecules to treat cell lineage specific disease. In the case of Parkinson's disease, for example, it is the loss of midbrain dopaminergic (DA) neurons that results in the appearance of disease symptoms. Thus, there is need for methods of producing DA neuronal cells from pluripotent cells, since such cells could be used both therapeutically and in disease models, e.g., to identify new therapeutics for treatments for neurodegenerative disease.

### SUMMARY OF THE INVENTION

The current disclosure provides for methods for differentiating stem and progenitor cells into neural cells through an approach that excludes the use of inhibitors of the BMP4 pathway resulting in SMAD inhibition. Aspects of the disclosure relate to a method for differentiating stem or progenitor cells into neural cells, the method comprising contacting the cells with a compound selected from DMH1, DMH2, K02288, A8301, or combinations thereof. The disclosure also describes a method for producing neural cells from stem or progenitor cells, the method comprising contacting the cells with a compound selected from DMH1, DMH2, K02288, A8301, or combinations thereof. Further aspects relate to a neural cell and a population of cells produced by the methods of the claims. Also provided is a method of treating a disease in a mammalian subject comprising administering to the subject a therapeutically effective number of neural cells or a population of cells made by methods of the disclosure. A further method aspect relates to a method of screening a test compound comprising: (a) contacting the test compound with cells of the disclosure; and (b) measuring the function, physiology, or viability of the cells.

Certain aspects relate to a method for differentiating stem or progenitor cells into neural cells, the method comprising culturing the cells in medium comprising a ROCK inhibitor for 2-48 hours, removing the medium and culturing the cells in medium comprising an ALK inhibitor for 1-10 days, wherein the ROCK inhibitor consists of Y27632 and the ALK inhibitor consists of a compound selected from DMH1, DMH2, K02288, A8301, or combinations thereof; and wherein the method excludes contacting the cells with a BMP4 inhibitor (such as Noggin, Chordin) or a SMAD inhibitor.

In some aspects, the compound consists of DMH1, DMH2, K02288, or A8301. In some aspects, the compound comprises or consists of DMH2. In some aspects, the compound consists of DMH1 and DMH2. In some aspects, the compound consists of K02288 and DMH2. In some aspects, the compound consists of A8301 and DMH2.

In some aspects, the stem or progenitor cells comprise induced pluripotent stem cells (iPSCs) or embryonic stem (ES) cells. In some aspects, the stem or progenitor cells comprise hematopoietic stem or progenitor cells. In some aspects, the stem or progenitor cells are totipotent, pluripotent, or multipotent stem cells. In a particular aspect, the cells comprise embryonic stem (ES) cells. In some aspects, the cells are human cells or are derived from human cells. In some aspects, the cells are human ES cells. In some aspects, the cells comprise HS420 cells.

In some aspects, contacting the cells comprises contacting the cells for a time period of about 1-7 days of substantially continuous contact. The term “substantially continuous contact” refers to a contact that is for at least 90, 91, 92, 93, 94, 95, 96, 97, 98, 99, or 100% of the time during a certain time period, but does not exclude brief periods of non-contact, such as periods in which the cells may be undergoing a washing, re-plating, trypsinization, or a change in the cell culture medium. In some aspects, the time period comprises at least, at most, about, or exactly 1, 2, 3, 4, 5, 6, or 7 days (or any derivable range therein).

In some aspects, the cells are contacted with 0.01-5 μM of a compound. In some aspects, the cells are contacted with at least, at most, about, or exactly 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, or 10 μM of a compound, or any derivable range therein. In some aspects, the cells are contacted with 0.2 μM of a compound.

In some aspects, the method further comprises contacting the cells with a Rho Kinase (ROCK) inhibitor. In some aspects, the ROCK inhibitor comprises Y27632. Other ROCK inhibitors useful in the aspects of the disclosure include Fasudil, Ripasudil, Netarsudil, RKI-1447, GSK429286A, and Y30141. In some aspects, the cells are contacted with 5-15 μM ROCK inhibitor. In some aspects, the cells are contacted with at least, at most, about, or exactly 1, 1.5, 2, 2.5, 3, 3.5, 4, 4.5, 5, 5.5, 6, 6.5, 7, 7.5, 8, 9, 10, 11, 12, 13, 14, 15, 16, 17, 18, 19, 20, 21, 22, 23, 24, 25, 30, 35, 40, 45, 50, 55, 60, 65, 70, 75, or 80 μM ROCK inhibitor (or any derivable range therein). In some aspects, the cells are contacted with the ROCK inhibitor prior to contact with the compound. In some aspects, the cells are contacted with the ROCK inhibitor immediately prior to contact with the compound. In some aspects, the contact with the ROCK inhibitor and the contact with the ALK inhibitor compound is overlapping for a time period. In some aspects, the contact with the ROCK inhibitor and the contact with the ALK inhibitor are non-overlapping and comprises a time period between when the cells are contacted with the ROCK inhibitor and when the cells are contacted with the ALK inhibitor. In some aspects, the cells are contacted with the ROCK inhibitor after the ALK inhibitor. In some aspects, the contact with the ROCK inhibitor and the contact with the ALK inhibitor are non-overlapping and comprises a time period between when the cells are contacted with the ALK inhibitor and when the cells are contacted with the ROCK inhibitor. In some aspects, the time period is at least, at most, or about 1, 2, 3, 4, 5, 10, 15, 30, or 45 min or 1, 2, 3, 4, 5, 6, 7, 8, 9, 10, 12, 18, or 24 hours, or 1, 2, 3, 4, 5, 6, 7, 8, 9, or 10 days (or any derivable range therein). In some aspects, the cells are contacted with the ROCK inhibitor for a time period of 1-48 hours. In some aspects, the cells are contacted with the ROCK inhibitor for a time period of 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or 2.5, 3, 3.5, 4, 4.5, 5, 5.5, 6, 6.5, or 7 days (or any derivable range therein).

In some aspects, the method excludes contacting the cells with a Smad inhibitor. In some aspects, the method excludes contacting the cells with LDN193189 and/or SB431542. In some aspects, the method excludes contacting the cells with LDN193189 and SB431542. In some aspects, the method excludes dual or mono-Smad inhibition. In some aspects, the method excludes contacting the cells with a Noggin protein or Noggin modulator, such as an activator or repressor of Noggin or a direct activator or repressor of Noggin. In some aspects, the method excludes contacting the cells with a BMP4 inhibitor. In some aspects, the method excludes contacting the cells with Noggin and/or Chordin or activators thereof.

In some aspects, the neural cells are further defined as dopaminergic neurons, glutamatergic, serotoninergic, cholinergic, GABAergic, motoneurons, astrocytes, or oligodendrocytes. In some aspects, the neural cells are further defined as a neural cell described herein. The term “dopaminergic neuron” or “DA neuron” refers to a neuron having an ability to produce dopamine (3,4-dihydroxyphenylethylamine). A dopaminergic neuron does not need to produce dopamine all the time, but only needs to have dopamine production capability. In aspects of the disclosure, the DA neuron may be a DA neuron of the A8 group, A9 group, A10 group, A11 group, A12 group, A13 group, A14 group, A15 group, A16 group, Aaq group, or telencephalic group.

In some aspects, contacting the cells with a compound comprises culturing the cells in a cell culture medium comprising the compound. In some aspects, the cell culture medium comprises one or more of DMEM medium, DMEMF12 medium, Neurobasal medium, antimicrobial agents, B-27 supplement, N-2 supplement and L-glutamine. In some aspects, the neural cells are further defined as Nestin+, Pax-6+, and/or Sox-1+ cells. In some aspects, the method comprises or further comprises selecting cells that are Nestin+, Pax-6+, and/or Sox-1+. In some aspects, Nestin+, Pax-6+, and Sox-1+ are selected. The methods of the disclosure may exclude contacting the cells with serum. According, aspects of the disclosure provide for the culturing of cells in serum-free medium. In some aspects, the cells are cultured on a substrate comprising laminin. Examples include Laminin 521. In some aspects, the cells are cultured on a substrate comprising polyornithine and/or Matrigel.

The cells may be plated at a density of, a density of at least, or a density of at most 1×10³, 2×10³, 3×10³, 4×10³, 5×10³, 6×10³, 7×10³, 8×10³, 9×10³, 1×10⁴, 2×10⁴, 3 λ10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, or 2×10⁶ cells/cm² (or any derivable range therein). In some aspects, the cells are plated at a density of 40000-60000 cells/cm². In some aspects, the cells are plated at a density of 50000 cells/cm². In some aspects, the cells are plated at a density of 5000-15000 cells/cm². In some aspects, the cells are plated at a density of 10000 cells/cm².

In some aspects, the percentage of non-neural cells in the cell culture after contact with the compound for a period of time is less than 30% in the population of cells of the current disclosure. In some aspects, the percentage of non-neural cells in the cell culture after contact with the compound for a period of time is less than 40%, 35%, 30%, 25%, 20%, 15%, 10%, 9%, 8%, 7%, 6%, 5%, 4%, 3%, 2%, or 1% in the population of cells (or any derivable range therein). In some aspects, the period of time is 4-8 days. In some aspects, the time period is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or any derivable range therein.

In some aspects of the disclosure, the disease comprises a neurodegenerative disease. In some aspects, the subject is a human subject. In some aspects, the subject is a mammal. In some aspects, the subject comprises a laboratory animal, pig, rat, goat, rabbit, cat, dog, horse, or mouse.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments or aspects described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments or aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the chemical structures of DMH1, DMH2, A8301, K02288, and ML347.

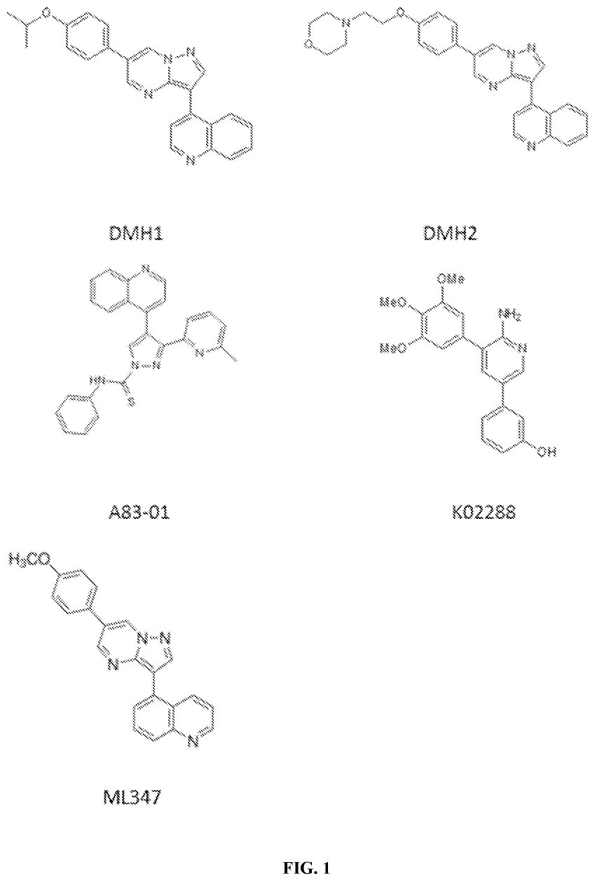

FIG. 2 depicts method embodiments detailing the timing and duration of administration of compounds as well as the cell culture medium and supplements used during each period.

FIG. 3: Morphological modifications of embryonic stem cells exposed to DMH1, DMH2, K 0228, or A8301 at 0.2 μM after one week. Also shown are the the morphological modifications of embryonic stem cells exposed to dual SMAD inhibition and to Noggin.

FIG. 4: Immunostaining against nestin, Pax-6, Sox-1 of cells exposed to ALKi for one week at the concentration of 0.2 uM. The following markers were used: (i) nestin and Sox-1 which are expressed in early neuro-epithelial cells rapidly after neural induction and (ii) Pax-6 which is a transcription factor expressed early in neuroepithelial cells and persistent only in forebrain.

FIG. 5: Quantification of neuroectodermal markers following exposition of HS420 cells one week with ALKi or dualSMAD inhibition. Results are expressed as the percentage of control condition (DMSO) in three independent experiments. The bars in each bar graph represent, from left to right, data from DMH-1, DMH-2, K02288, A8301, and LDN+SB.

FIG. 6: HS420 cells were exposed for 8 days to dual SMAD inhibition or ALK inhibitors at the concentration of 0.2 μM. Regionalization markers were measured by Q-RT-PCR and normalized with control conditions (no compounds, vehicle alone).

FIG. 7: Neuronal maturation f HS420 cells (one month) exposed to ALKi versus dualSMAD inhibition.

### DETAILED DESCRIPTION OF THE INVENTION

Formation of neuroectoderm is a crucial step in the differentiation of pluripotent stem cells towards neural cells and tissues. The current disclosure provides an effective alternative to methods that use a Smad inhibition protocol (Noggin, LDN193189, and SB431542 in various combinations).

### I. Definitions

The term “differentiation” as used with respect to cells in a differentiating cell system refers to the process by which cells differentiate from one cell type (e.g., a multipotent, totipotent or pluripotent differentiable cell) to another cell type such as a fully differentiated cell. More generally, the term “differentiation” refers to a process whereby an unspecialized stem cell or a precursor cell acquires the features of a specialized or fully differentiated cell such as a brain, heart, liver, or muscle cell. Differentiation is controlled by the interaction of a cell's genes with the physical and chemical conditions outside the cell, usually through signaling pathways involving proteins embedded in the cell surface.

Progenitor cells in this disclosure are included within somatic cells. Progenitor cells are multipotent cells. Totipotent, pluripotent, and multipotent cells can be “stem cells,” which are capable of differentiating into one or more different cell types. The term “stem cells,” “embryonic stem cell”, “induced pluripotent stem cell” have been described above.

In the present specification, the “stem cell” refers to a cell that can be cultured in vitro and can be differentiated into cells of plural lineages constituting the body. It specifically includes ES cell, pluripotent stem cell derived from fetal primordial germ cell (EG cell: Proc Natl Acad Sci USA. 1998, 95: 13726-31), pluripotent stem cell derived from testis (GS cell: Nature. 2008, 456: 344-9), induced pluripotent stem cell derived from somatic cell (induced pluripotent stem cells; iPS cell), and human pluripotent somatic stem cell (neural stem cell), preferably iPS cell and ES cell, more preferably iPS cell.

### II. Cell Sources

Aspects of the disclosure relate to the differentiation of a starting population of stem or progenitor cells into neural cells. The stem or progenitor cells may be one described herein and/or derived from a source described herein. In some aspects, the stem or progenitor cell is an ES cell. It is contemplated that an ES cell derived from any warm-blooded animal, preferably mammal can be used. Examples of the mammal include mouse, rat, guinea pig, hamster, rabbit, cat, dog, sheep, swine, bovine, horse, goat, monkey, and human. Preferable examples of the ES cell include ES cells derived from human. In some aspects, the stem or progenitor cell excludes a cell derived from or taken from a human fetus.

Specific examples of the ES cell include an ES cell of a mammal and the like, which has been established by culturing an early embryo prior to implantation, an ES cell established by culturing an early embryo prepared by nucleus transplantation of the nucleus of a somatic cell, and an ES cell obtained by alteration of a gene on the chromosomes of these ES cells by a genetic engineering method. Each ES cell can be prepared according to a method generally performed in the pertinent field, or a known document.

Mouse ES cell was established in 1981 by Evans et al (1981, Nature 292: 154-6) and Martin G R. et al. (1981, Proc Natl Acad Sci 78: 7634-8) and can be purchased from, for example, Sumitomo Dainippon Pharma Co., Ltd. (Osaka, Japan) and the like.

Human ES cell was established in 1998 by Thomson et al (Science, 1998, 282: 1145-7), and is available from WiCell Research Institute (website: http://www.wicell.org/, Madison, Wis., USA), US National Institute of Health, Kyoto University and the like and can be purchased from, for example, Cellartis (website: http://www.cellartis.com/, Sweden) and the like.

In some aspects, the stem or progenitor cell is an iPSC (also known as iPS cell). As an iPS cell, an iPS cell derived from any warm-blooded animal, preferably mammal, can be used. Examples of the mammal include mouse, rat, guinea pig, hamster, rabbit, cat, dog, sheep, swine, bovine, horse, goat, monkey, and human. Preferable examples of the iPS cell include an iPS cell derived from human.

Specific examples of the iPS cell include a cell that acquired multipotency as in ES cell, which can be obtained by introducing plural genes into a somatic cell such as skin cell and the like. For example, an iPS cell obtained by introducing Oct3/4 gene, Klf4 gene, c-Myc gene and Sox2 gene, and an iPS cell obtained by introducing Oct3/4 gene, Klf4 gene and Sox2 gene (Nat Biotechnol 2008; 26: 101-106). Other than these, a method of further decreasing transgene (Nature. 2008 Jul. 31; 454 (7204): 646-50), a method utilizing a low-molecular-weight compound (Cell Stem Cell. 2009 Jan. 9; 4(1): 16-9, Cell Stem Cell. 2009 Nov. 6; 5(5): 491-503), a method utilizing a transcription factor protein instead of gene (Cell Stem Cell. 2009 May 8; 4(5): 381-4) and the like. The produced iPS cell can be used for the present invention irrespective of the production method thereof.

Examples of the human iPS cell line include, specifically, 253G1 strain (iPS cell line prepared by expressing OCT4/SOX2/KLF4 in skin fibroblast of 36-year-old female), 201B7 strain (iPS cell line prepared by expressing OCT4/SOX2/KLF4/c-MYC in skin fibroblast of 36-year-old female), 1503-iPS (297A1) (iPS cell line prepared by expressing OCT4/SOX2/KLF4/c-MYC in skin fibroblast of 73-year-old female), 1392-iPS (297F1) (iPS cell line prepared by expressing OCT4/SOX2/KLF4/c-MYC in skin fibroblast of 56-year-old male), NHDF-iPS (297 L1) (iPS cell line prepared by expressing OCT4/SOX2/KLF4/c-MYC in skin fibroblast of newborn boy) and the like.

### III. Cell Culture Method

Aspects of the disclosure include methods for differentiating a stem or progenitor cell or a starting population of stem and/or progenitor cells into neural cells. The methods of the disclosure may comprise or further comprise culturing the cells in a medium as defined herein and/or comprising or further comprising supplements and components described herein. These aspects are discussed in more detail below. It is further specifically contemplated that the methods of the disclosure may exclude culturing the cells in medium or with supplements described herein.

In methods of the disclosure, the cells are generally cultured on a culture vessel. Examples of the culture vessel to be used here include flask, tissue culture flask, dish, petri dish, tissue culture dish, multi dish, microplate, microwell plate, multiplate, multiwell plate, chamber slide, petri dish, tube, tray, culture bag, and roller bottle. The culture vessel may comprise a coating suitable for maintenance and culture of stem cells. In some aspects, the culture vessel comprises a coating of laminin 521. In some aspects, a culture vessel may be coated with a feeder cell or an extracellular substrate component. In some aspects, the feeder cell comprises fibroblasts, such as mouse embryonic fibroblast (MEF), mouse fibroblast (STO), and the like. The feeder cell may be inactivated by a method known in the art, such as by irradiation, gamma ray radiation, or treatment with an anti-cancer agent (e.g. mitomycin C). Examples of the extracellular substrate component include fibrous protein such as gelatin, collagen, elastin, glucosaminoglycan and proteoglycan such as hyaluronic acid, chondroitin sulfate, cell adhesive protein such as fibronectin, vitronectin, laminin, laminin 521, and basal lamina component such as Matrigel.

The cell culture medium may be one that is known and used in the art for culturing stem cells, such as STEMFLEX cell culture medium. In some aspects, the medium comprises a medium to culture neurons, such as NEUROBASAL medium, NEUROBASAL-A medium, or Neural Progenitor Basal medium. In some aspects, the medium comprises NS-A medium, BME medium, BGJb medium, CMRL 1066 medium, Glasgow MEM medium, Improved MEM Zinc Option medium, IMDM medium, Medium 199 medium, Eagle MEM medium, aMEM medium, DMEM medium, DMEM/F12 medium, ham medium, RPMI 1640 medium, Fischer's medium, and mixed medium thereof and the like. The cell culture mediums are generally available for purchase from Invitrogen, SIGMA, Wako Pure Chemical Industries, Ltd., Sumitomo Dainippon Pharma Co., Ltd. and the like.

The medium to be used in this differentiation method may be a serum-containing medium or a serum-free medium (such as KNOCKOUT medium). As used herein, the serum-free medium means a medium free of a non-adjusted or unpurified serum, and a medium containing purified blood-derived components and animal tissue-derived components (e.g., growth factor) corresponds to a serum-free medium. When the medium to be used in this differentiation method is a serum-containing medium, and a serum of a mammal such as fetal bovine serum and the like can be used as the serum. The concentration of the serum in the medium is generally 0.01-20 wt % or 0.1-10 wt %.

The medium to be used in this differentiation method may also contain a serum replacement. Examples of the serum replacement include albumin (e.g., lipid-rich albumin), transferrin, fatty acid, collagen precursor, trace element (e.g., zinc, selenium), B-27 supplement, N2 supplement, Replacement KnockOut serum replacement, 2-mercaptoethanol, 3′thiolglycerol, and equivalents thereof. The concentration of these in the media is the same as the concentration of the aforementioned serum in the medium.

The medium to be used in method aspects of the disclosure may also contain lipid, amino acid (e.g., non-essential amino acid), vitamin, growth factor, cytokine, antioxidant, 2-mercaptoethanol, pyruvic acid, buffering agent, inorganic salt, antibiotic (e.g., penicillin and streptomycin) or antibacterial agent (e.g., amphotericin B) and the like. The concentration of these in the media is the same as the concentration of the aforementioned serum in the medium.

Other culture conditions such as culture temperature, CO₂ concentration and the like can be appropriately determined. While the culture temperature is not particularly limited, it is, for example, about 30-40° C., preferably about 37° C. The CO₂ concentration is, for example, about 1-10%, preferably about 5%.

Further aspects of the disclosure include the evaluation of cells, for example, an evaluation method of expression of protein by utilizing an antigen-antibody reaction, an evaluation method of gene expression by utilizing quantitative RT-PCR, and the like. Aspects of the disclosure include evaluating the cells for expression of a cell marker, such as Pax6, Otx2, FoxA2, Lmxla, and Msx1.

Cell culture conditions may be provided for the culture of neural or progenitor cells as provided herein. In certain aspects, starting cells of a selected population may comprise at least or about 10⁴, 10⁵, 10⁶, 10⁷, 10⁸, 10⁹, 10¹⁰, 10¹¹, 10¹², 10¹³ cells or any range derivable therein. The starting cell population may have a seeding density of at least or about 10, 10¹, 10², 10³, 10⁴, 10⁵, 10⁶, 10⁷, 10⁸ cells/ml, or any range derivable therein.

A culture vessel used for culturing the cells of the disclosure, or progeny cells thereof, can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing the cells therein. The cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain aspect, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, laminin, laminin 521, fibronectin, and mixtures thereof for example Matrigel™, and lysed cell membrane preparations.

### IV. Neural Cells

Aspects of the disclosure relate to the differentiation of cells into neural cells. The methods may be used to generate one or more types of neural cells including motor neurons, sensory neurons, and interneurons. A typical neuron consists of a cell body (referred to as a soma), dendrites, and an axon. The methods may be used to generate cholinergic neurons, GABAergic neurons, motor neurons, astrocytes, oligodendrocytes, glutamatergic neurons, dopaminergic neurons, and/or serotonergic neurons. In particular aspects, the methods are used to generate dopaminergic neurons. In some aspects, the methods include differentiating neural cells into neurons, astrocytes, oligodendrocytes, dopamine neurons, or motor neurons, pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, and cholinergic neurons.

Aspects of the disclosure also include the evaluation of progenitor and/or neural cells. For example, the production or differentiation of neural cells in a cell population may be determined through the presence of certain cell markers. Those markers may vary depending on the species or organism that is used for the starting population. Examples of neural cell markers in organisms such as humans include transcription factors or structural proteins. Examples of transcription factors include MYT1L, BRN2, SOX1, PAX6, NKX6.1, OLIG2, NGN2, LHX3, ISL1/2, and HB9. Other neural markers include tubulin (e.g., Tubb2a and Tubb2b), Map2, Synapsin (e.g., Syn1 and Syn2), synaptophysin, synaptotagmins (e.g., Sytl, Syt4, Syt13, Syt 16), NeuroD, cholineacetyltransferase (ChAT) (e.g., vesicular ChAT), neurofilament, neuromelanin, Tuj 1, Thy1, Chat, GluR (kainite 1), Neurod 1, and the like. Expression of receptors for excitatory and inhibitory neurotransmitters can also be used to assess the number and quality of neural cells generated. In addition, gross cell morphology may be used to identify neural cells in a population of non-neural cells. The neural cells of the disclosure may also exclude one or more of the markers listed herein such as MYT1L, BRN2, SOX1, PAX6, NKX6.1, OLIG2, NGN2, LHX3, ISL1/2, and HB9, Tubb2a, Tubb2b, Map2, Synapsin, Syn1, Syn2, synaptophysin, synaptotagmins, Sytl, Syt4, Syt13, Syt 16, NeuroD, cholineacetyltransferase (ChAT), vesicular ChAT, neurofilament, neuromelanin, Tuj 1, Thy1, Chat, GluR (kainite 1), and Neurod 1.

The presence of neural cells may also be assessed functionally. For example, the cells may be assessed according to electrophysiological characteristics. These assessments may be made using patch-clamp recordings. Other functional characteristics include ability to fire action potentials, produce an outward current in response to glycine, GABA or kainite, and produce an inward current in response to glutamate.

Neural cells may be assessed and thus identified by the presence of one or more, including 2, 3, 4, 5, or more, of any of the foregoing characteristics and/or markers.

The neural cells or cell population may also be assessed for expression of markers characteristic of the non-neural starting cell population. Reprogramming, in some instances, may be evaluated by the increased expression of neural markers and decreased expression of markers of the non-neural starting cells.

In some aspects, the neural cell is further defined as a dopaminergic (DA) neuron. DA neurons can be confirmed by evaluating the expression variation of proteins and genes that are specifically expressed by the dopaminergic neuron (in the present specification, the above-mentioned proteins and genes are sometimes referred to as a dopaminergic neuron marker). The above-mentioned evaluation of expression variation of dopaminergic neuron cell marker can be performed by, for example, an evaluation method of expression of protein by utilizing an antigen-antibody reaction, an evaluation method of gene expression by utilizing quantitative RT-PCR and the like. Examples of the above-mentioned dopaminergic neuron cell marker, which is present in the midbrain, include tyrosine hydroxylase (TH), OTX2, FOXA2, LMX1A, LMX1B, PITX3, EN1 and NURR1 gene/protein.

In addition, whether the dopaminergic neuron obtained by the production method of the present invention has functions equivalent to those of dopaminergic neuron in vivo can be confirmed by evaluating dopamine release, and responsiveness to oxidative stress and drug stimulation.

The cells obtained during the processes of the production method of the present invention and the dopaminergic neuron of the present invention can be cryopreserved and thawed. Freezing and thawing methods of cells are known in the pertinent field, and are not particularly limited as long as they do not influence differentiation potency, viability, dopamine production capability and the like of the cells. For example, the dopaminergic neuron of the present invention can be preserved at −80° C. by washing cells with PBS, detaching same from a culture dish with a cell-dispersion solution (e.g., Accutase (registered trade mark) Innovative Cell Technologies), removing the cell-dispersion solution, and suspending the cells in a cryopreservation solution (e.g., cell banker 2 (LSI Medience Corporation)). Examples of the thawing method include a method comprising thawing in a thermostatic tank at 37° C., washing a cryopreservation solution by centrifugation, and suspending in a medium for use, and the like. When the cells obtained during the processes of the production method of the present invention are frozen and thawed, Nurrl positive dopaminergic neuron can also be induced from the cells after thawing.

### V. Methods of Use of the Neural Cells

Methods of the disclosure relate to the production of a neural progenitor cells that may be, used for treatment of subjects. For example, the cells produced by methods of the disclosure may be used to treat neurodegenerative diseases. Non-limiting examples of neurodegenerative diseases include Alzheimer disease; epilepsy; Huntington's Disease; Parkinson's Disease; stroke; spinal cord injury; traumatic brain injury; Lewy body dementia; Pick's disease; Niewmann-Pick disease; amyloid angiopathy; cerebral amyloid angiopathy; systemic amyloidosis; hereditary cerebral hemorrhage with amyloidosis of the Dutch type; inclusion body myositis; mild cognitive impairment; Down's syndrome; and neuromuscular disorders including amyotrophic lateral sclerosis (ALS), multiple sclerosis, and muscular dystrophies including Duchenne dystrophy, Becker muscular dystrophy, Facioscapulohumeral (Landouzy-Dejerine) muscular dystrophy, and limb-girdle muscular dystrophy (LGMD). Also included is neurodegenerative disease due to stroke, head trauma, spinal injury, or other injuries to the brain, peripheral nervous, central nervous, or neuromuscular system. Certain aspects of the methods set forth herein pertain to methods of preventing a disease or health-related condition in a subject.

The current disclosure provides a medicament containing a neuron produced by the methods of the disclosure. As used herein, the neuron is not particularly limited as long as it is a cell obtained by the above-mentioned production method of the disclosure.

In this medicament, a neuron may be used as is, or as a cell aggregate obtained by concentration by passing through a filter and the like, such as pellet and the like. Furthermore, the medicament can also be added with a protector such as DMSO (dimethyl sulfoxide) and the like and cryopreserved. For safer utilization of the medicament, the medicament may be subjected to a treatment under such conditions as to retain the function of the neuron and denature pathogenic protein, for example, heat treatment, radiation treatment and the like. Moreover, to prevent growth of the neuron in an amount more than necessary, the medicament may be subjected to, in combination with the above-mentioned treatments, suppression of growth by a mitomycin C pre-treatment and the like, and a treatment by a method including introducing a gene of a metabolic enzyme naturally absent in mammals into the neurons, administering an agent in an inactivated form as necessary to allow for the agent to be converted to a toxicant only in the neurons, into which the gene of a metabolic enzyme naturally absent in mammals has been introduced, thus leading the cells to eradication (suicide gene therapy) and the like.

Since the medicament of the current disclosure is safe and has low toxicity, it can be administered to a mammal (e.g., human, mouse, rat, guinea pig, swine, monkey).

In some aspects, a neural cell may be prepared using a patient's own cell or a cell of a donor having a histocompatibility type in a tolerable range is used for the medicament of the current disclosure. When sufficient cells cannot be obtained due to age, constitution and the like, the cells embedded with a polyethylene glycol or silicon capsule, a porous container and the like can also be transplanted to avoid rejection. The dose (amount to be transplanted) and administration frequency (number of times to be transplanted) of the medicament of the present disclosure can be appropriately determined according to the age, body weight, symptom and the like of the patients who receive administration.

In some aspects, a medicament containing the neuron of the disclosure can efficiently engraft in the body of patients by administration (transplantation) thereof, which in turn enables efficient production (release) of dopamine in the body of patients. Therefore, the medicament of the disclosure is useful for the treatment of diseases caused by decreased production (release) of dopamine, for example, neurodegenerative diseases such as Parkinson's disease, Huntington chorea, Alzheimer's disease, epilepsy and schizophrenia and the like.

In some aspects, the neural cells of the disclosure may be used in a method for screening for a drug compound, such as a compound for the treatment of neurodegenerative diseases. For example, whether the test compound is useful as a medicament can be evaluated by contacting the test compound alone or in combination with other medicament with the neuron of the disclosure, and measuring morphological or functional change of the neuron. Examples of the method for measuring the functional change include measuring the amount of dopamine produced or released from the neuron. In some aspects, the dopaminergic neuron is preferably a cell showing the same phenotype as the disease to be the treatment target, and particularly preferred is a dopaminergic neuron produced by inducing differentiation of a stem cell produced from a somatic cell derived from the disease.

Examples of the test compound include peptide, protein, antibody, nonpeptidic compound, synthetic compound, fermentation product, cell extract, plant extract, animal tissue extract, plasma and the like. As used herein, the test compound may form a salt. As the salt, a salt with a physiologically acceptable acid (e.g., inorganic acid, organic acid), a base (e.g., alkali metal salt, alkaline earth metal salt, aluminum salt) and the like is used, and examples of such salt include a salt with an inorganic acid (e.g., hydrochloric acid, phosphoric acid, hydrobromic acid, sulfuric acid), a salt with an organic acid (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid), sodium salt, potassium salt, calcium salt, magnesium salt, barium salt, and aluminum salt can be used.

The medicament obtained using the above-mentioned screening can be formulated using a physiologically acceptable additive and according to a known method.

### VI. Additional Agents

It is contemplated that methods of the disclosure include the administration of additional agents. In some aspects, the additional agent comprises one or more BMP inhibitors such as Noggin, chordin, dorsomorphin, LDN193189 (4-(6-(4-(piperazin-1-yl)phenyl)pyrazolo[1,5-a]pyrimidin-3-yl)quinoline hydrochloride), dorsomorphin(6-[4-(2-piperidin-1-ylethoxy)phenyl]-3-pyridin-4-ylpyrazolo[1,5-a]pyrimidine) and the like; TGFβ family inhibitors such as SB431542 (4-[4-(1,3-benzodioxol-5-yl)-5-(2-pyridinyl)-1H-imidazol-2-yl]-benzamide), A8301 (3-(6-methylpyridin-2-yl)-1-phenylthiocarbamoyl-4-quinolin-4-ylpyrazole) and the like; GSK30 inhibitors such as CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), and BIO (6-bromo-indirubin-3′-oxime); Smoothened agonists such as purmorphamine (N-(4-morpholinophenyl)-2-(1-naphthyloxy)-9-cyclohexyl-9H-purin-6-amine), and SAG (N-methyl-N′-(3-pyridinylbenzyl)-N′-(3-chlorobenzo[b]thiophene-2-carbonyl)-1,4-diaminocyclohexane); and specification factors such as Sonic hedgehog (SHH) and fibroblast growth factor-8 (FGF-8). In some aspects of the disclosure, the methods exclude contact of the cells with one or more of the additional agents described herein. In some aspects, the methods exclude contact of the cells with one or more BMP inhibitors such as chordin, dorsomorphin, Noggin, LDN193189 (4-(6-(4-(piperazin-1-yl)phenyl)pyrazolo[1,5-a]pyrimidin-3-yl)quinoline hydrochloride), dorsomorphin(6-[4-(2-piperidin-1-ylethoxy)phenyl]-3-pyridin-4-ylpyrazolo[1,5-a]pyrimidine) and the like; TGFβ family inhibitors such as SB431542 (4-[4-(1,3-benzodioxol-5-yl)-5-(2-pyridinyl)-1H-imidazol-2-yl]-benzamide); GSK30 inhibitors such as CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), and BIO (6-bromo-indirubin-3′-oxime); Smoothened agonists such as purmorphamine (N-(4-morpholinophenyl)-2-(1-naphthyloxy)-9-cyclohexyl-9H-purin-6-amine), and SAG (N-methyl-N′-(3-pyridinylbenzyl)-N′-(3-chlorobenzo[b]thiophene-2-carbonyl)-1,4-diaminocyclohexane); and growth factors such as Sonic hedgehog (SHH) and fibroblast growth factor-8 (FGF8).

In some aspects, the additional agent includes one or more of an activator of the phosphatidylinositol 3-kinase signaling pathway and an activator of the MAPK signaling pathway. In some aspects, the additional agent excludes one or more of an activator of the phosphatidylinositol 3-kinase signaling pathway and an activator of the MAPK signaling pathway. In some aspects, the additional agent can comprise one or more of Midkine, Pleiotrophin, insulin-like growth factor-1, an inhibitor of the TGF-β superfamily signaling pathway, A8301, SB431542, dorsomorphin, an inhibitor of the Wnt signaling pathway, PNU-74654, Dickkopf, an activator of the Notch signaling pathway such as Delta-1, Delta-2, Delta-3, Delta-4, Jagged-1, Jagged-2, an activator of the protein kinase signaling pathway such as Forskolin, or dibutyryl cAMP, an activator of tyrosine kinase anaplastic lymphoma kinase (ALK), and activator of insulin-like growth factor (IGF) receptor, and inhibitor of SMAD2, SMAD3, SMAD4, SMAD1, SMAD5, SMAD8, an inhibitor of Wnt or LRP binding to Frizzled, or an inhibitor of β-catenin stabilization. In some aspects, the methods exclude contact of cell aspects described herein with one or more of an activator of the phosphatidylinositol 3-kinase signaling pathway and an activator of the MAPK signaling pathway. In some aspects, the additional agent excludes one or more of an activator of the phosphatidylinositol 3-kinase signaling pathway and an activator of the MAPK signaling pathway. In some aspects, the additional agent can comprise one or more of Midkine, Pleiotrophin, insulin-like growth factor-1, an inhibitor of the TGF-β superfamily signaling pathway, SB431542, an inhibitor of the Wnt signaling pathway, PNU-74654, Dickkopf, an activator of the Notch signaling pathway such as Delta-1, Delta-2, Delta-3, Delta-4, Jagged-1, Jagged-2, an activator of the protein kinase signaling pathway such as Forskolin, or dibutyryl cAMP, an activator of tyrosine kinase anaplastic lymphoma kinase (ALK), and activator of insulin-like growth factor (IGF) receptor, and inhibitor of SMAD2, SMAD3, SMAD4, SMAD1, SMAD5, SMAD8, an inhibitor of Wnt or LRP binding to Frizzled, or an inhibitor of β-catenin stabilization.

### VII. EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Inhibitors of Tyrosine Receptor Kinases to Drive Neural Differentiation of Pluripotent Stem Cells

Pluripotent Stem cells (PSCs) derived from embryos or induced from somatic cells have the capacity to differentiate into a wide variety of cell types of interest for tissue modelling and cell therapy. Mimicking embryonic development in vitro provide the best approach for generating differentiated cells with defined properties. The first natural specification of embryonic tissues occurs at the gastrulation stage, with the differentiation of the three germ layers ectoderm, mesoderm and endoderm from which all the adult tissues will derive.

Multiple combinations between Noggin, LDN193189 and SB431542 are commonly used and generally called “dualSMAD inhibition”. It represents to date the most widely used way to induce early neural specification of PSC in vitro. Described herein is the report of a differentiation approach that excludes “dual Smad inhibition,” and instead achieves differentiation through the use of 5 chemicals inhibiting ALK to induce neural specification of pluripotent stem cells in vitro: ML347, DMH1, DMH2, K02288 and A8301 (Table 1). Table 1 indicates their name, PubChem ID and toxicity on the human embryonic Pluripotent Stem Cell line (ePSC) HS420, by using an ATP-based cytotoxicity assay. The Cmax was defined as the maximal concentration in vitro allowing 100% of viability of cells.

TABLE 1
List and names of the 5 ALKi tested. The Cmax was defined as the in vitro
concentration allowing 100% of survival by sing an ATP quantification assay

| PubChem | Name | IUPAC Name | Cmax (μM) |
|---|---|---|---|
| 44577753 | ML347 | 5-[6-(4 Methoxyphenyl)pyrazolo[1,5- | 0.58 ± 0.20 |
| | | a]pyrimidin-3-yl]quinoline | |
| 50997747 | DMH1 | 4-[6-(4-Propan-2-yloxyphenyl)pyrazolo[1,5- | 0.97 ± 0.46 |
| | | a]pyrimidin-3-yl]quinoline | |
| 50997748 | DMH2 | 4-(2-(4-(3-(Quinolin-4-yl)pyrazolo[1,5-a]pyrimidin- | 0.58 ± 0.20 |
| | | 6-yl)phenoxy)ethyl)morpholine | |
| 46173038 | K02288 | 3-[6-Amino-5-(3,4,5-Trimethoxyphenyl)pyridin-3- | 0.53 ± 0.25 |
| | | Yl]phenol | |
| 16218924 | A8301 | 3-(6-Methylpyridin-2-yl)-N-phenyl-4-(quinolin-4- | 5.18 ± 1.78 |
| | | yl)-1H-pyrazole-1-carbothioamide | |
| 25195294 | LDN193189 | 4-(6-(4-(Piperazin-1-yl)phenyl)pyrazolo[1,5- | N/A |
| | | alpyrimidin-3-yl)quinoline | |
| 4521392 | SB431542 | 4-(4-(Benzo[d][1,3]dioxol-5-yl)-5-(pyridin-2-yl)-1H- | N/A |
| | | imidazol-2-yl)benzamide | |

A. Material and Methods
1. Chemicals and antibodies

LDN193189 was provided by Axonmedchem (ref 1509), stored at −20° C. at the concentration of 5 mM in DMSO. SB431542 was provided by Abcam (ref ab120163), stored at −20° C. at the concentration of 50 mM in DMSO. Noggin was provided by Miltenyi Biotec (ref 130-103-456), stored at −20° C. at the concentration of 100 g/ml in water. DMH1, DMH2, K02288 and A8301 were provided by Tocris (refs 4126, 5580, 4986 and 2939, respectively), stored at −20° C. at the concentration of 10 mM in DMSO. For immunostainings, the following fluorescent antibodies were used: Goat anti-Pax-6 (Santa Cruz), goat anti-Sox-1 (Santa Cruz), rabbit antinestin (Millipore), mouse anti-βIII-tubulin (Sigma), goat-anti-mouse IgG—Alexa 555 (Life technologies), donkey anti-goat IgG—Alexa 488 (Life technologies), goat anti-rabbit IgG —Alexa 488 (Life technologies).
2. Embryonic Stem Cell Culture and Differentiation

The human embryonic stem cell line HS420 (Gift from Dr Outi Hovatta, Karolinska institute, Sweden) was cultured in Stemflex medium (Thermofisher) on laminin 521-coated tissue culture flasks (Thermofisher) according manufacturer's instructions.

For neural differentiation, HS420 cells at 50% of confluency were passaged in new laminin 521-coated tissue culture plates at the density of 10000 cells/cm² in Stemflex medium supplemented with ROCK inhibitor (Y27632, abcam) at 10 μM. After 24 h, cells were submitted to a medium change, the Stemflex being replaced by the following neural induction medium: Neurobasal (Thermofisher) supplemented with penicillin/streptomycin 100 U/ml (Invitrogen), B-27 supplement (Thermofisher), L-glutamine 2 mM (Thermofisher). Cells were passaged at day 8 and subsequent confluency (every weeks), by the use of Accutase (Thermofisher) and replated on polyornithine/Matrigel (Sigma)-coated tissue culture flasks at the density of 50 000/cm².
3. Immunocytochemistry

Cells on glass coverslips were fixed with 1 mL of 0.5% paraformaldehyde in PBS for 30 minutes at room temperature. Cells were then washed three times with 1 mL of PBS and incubated overnight with primary antibodies in PBS containing 0.1% Triton X-100 (Sigma), and 1% bovine serumalbumine (Sigma). Cells were washed three times with 1 mL of PBS before incubation for 1 h30 min with the secondary antibody in PBS containing 0.1% Triton X-100 and 1% bovine serumalbumine. Cells were washed three times with PBS before exposure to DAPI 300 nM in PBS (Sigma) for 15 minutes at room temperature. After three washes in PBS, cells were rinsed with water and mounted in glass slides using Fluorsave reagent (Merck Millipore).
4. Quantitative RT PCR

Quantitative PCR was performed with Tecan Freedom Evo by using SYBR green fluorescence. cDNA was produced from RNA by using the prime script RT reagent kit (Takara). All primers were diluted at a concentration of 0.86 μM, and a master mix was used (Power SYBR Green Master Mix, Thermo Fisher Scientific).

| Gene | | Primer Sequence | SEQ ID NO: |
|---|---|---|---|
| Pax6 | Forward | ACATCTGGCTCCATGTTGGG | 1 |
| | Reverse | GGCAGCATGCAGGAGTATGA | 2 |
| Sox1 | Forward | AATACTGGAGACGAACGCCG | 3 |
| | Reverse | AACCCAAGTCTGGTGTCAGC | 4 |
| Nestin | Forward | GTTGACCAACGCTGGCGGGA | 5 |
| | Reverse | CGGGCTACCTCCTCCGTCGT | 6 |

B. Results

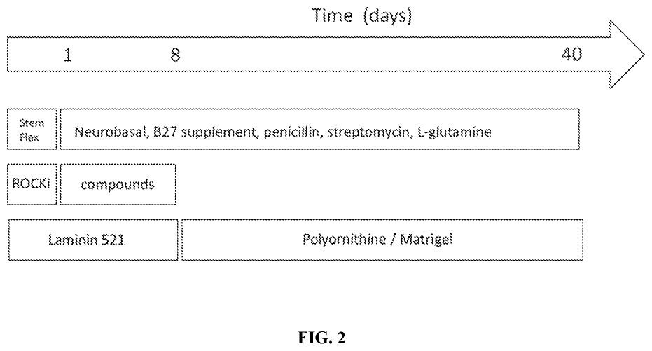

To study the effects of ML347, DMH1, DMH2, K02288 and A8301 on PSC, the human embryonic stem cell line HS420 was used. Cells were plated at a low density on laminin 521 (10000/cm²) in their maintenance medium (Stemflex) and exposed 24 h later to ML347, DMH1 or DMH2 or K02288 or A8301, during one week, at the concentration of 0.2 μM in a minimal medium suited for early neural induction. Once confluent (after 8 days), cells were passaged and plated in the same medium, without neural inductors, at the density of 50000/cm² polyornithine/Matrigel—coated tissue culture flasks for maturation (FIG. 2).

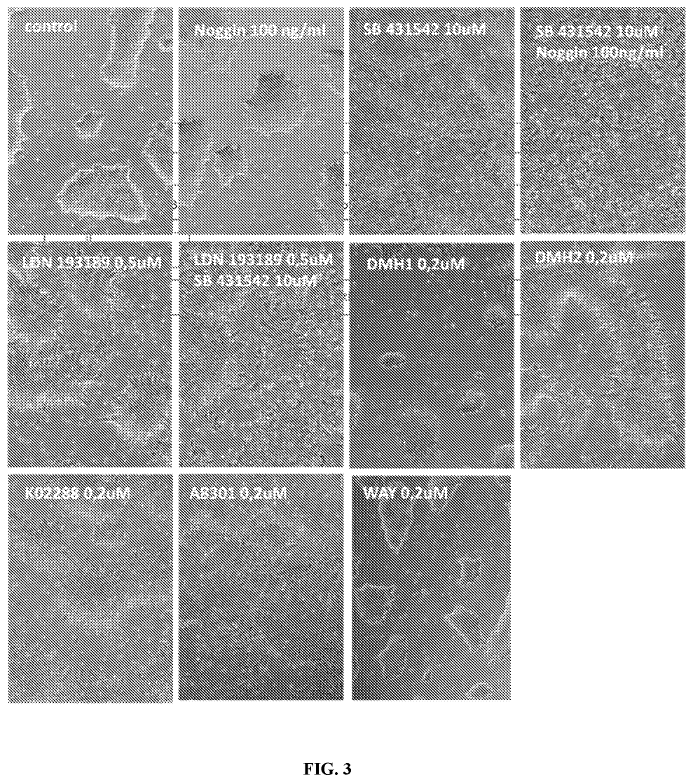

After one-week, embryonic stem cells exposed to ML347 showed large crystals in the culture (not shown), excluding he possibility to use this compound for any neural induction strategy. DMH1, DMH2, K 0228, or A8301 at 0.2 μM showed morphological modifications. In contrast to control conditions (DMSO) or a control compound activating Wnt signalling (WAY) where HS420 cells grew as compact, distinct and refringent colonies (FIG. 3), cells exposed to the four compounds showed flatter colonies with less refringence and epithelial-like cells at their periphery (FIG. 3). These epithelial-like cells notably invaded as a monolayer the free tissue culture plate between the colonies after exposure with K02288 and A8301, suggesting that the compounds favoured early neuroectodermal differentiation of embryonic stem cells. Similar morphological observations were done with SB431542 at 10M, LDN193189 at 0.5 μM in combination or not, in contrast to Noggin alone used at the concentration of 100 ng/ml (FIG. 3).

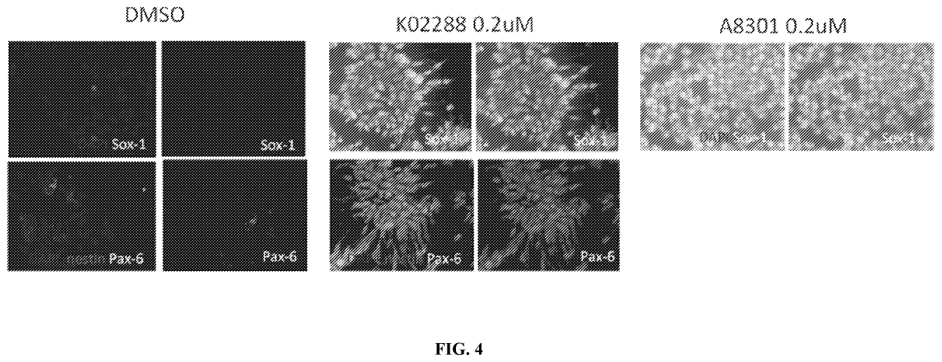

Immunofluorescent staining of flat cells showed different features of cells in the presence of the 4 compounds at day 8. The following markers were used: (i) nestin and Sox-1 which are expressed in early neuro-epithelial cells rapidly after neural induction (ii) Pax-6 which is a transcription factor expressed early in neuroepithelial cells and persistent only in forebrain. In control conditions, colonies were heterogenous for nestin, Sox-1 and Pax-6 staining, which a are common markers used for the identification of early ectodermal cells (FIG. 4). If some colonies were nestin+ Pax-6+ and expressed Sox-1 (not shown), a majority of cells in the culture did not express these markers, indicated that non-neural cells were growing in these conditions. In the presence of K02288 or A8301 at 0.2 μM, the number a high number of flat cells growing in monolayer homogeneously expressed nestin, Pax-6 and Sox-1, confirming the potent neural-inducing effects of this compounds (FIG. 4).

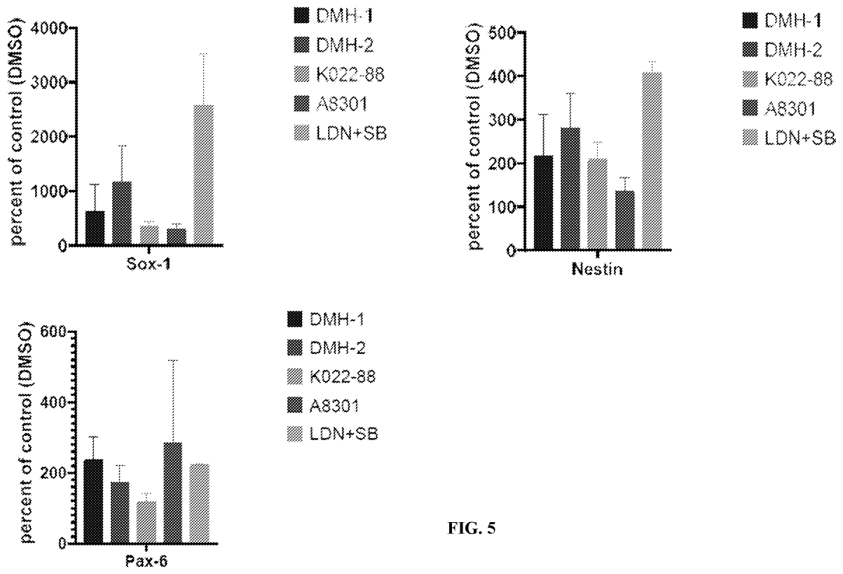

Q-RT-PCR experiments were performed to provide a quantitative description of neural-inducing effects of the 4 compounds (in three independent experiments). At day 8, cells exposed to LDN193189 at 0.5 μM increased the expression of Pax-6, Sox-1 and nestin mRNAs compared to control conditions (FIG. 5), confirming the up-regulation of neuro-ectodermal cells induction. SB431542 alone at 10 μM did not increase the three markers, with only a moderate increase of Sox-1. Similarly, Noggin alone at the concentration of 100 ng/ml had a moderate effect. The dual-SMAD inhibition obtained by the combination of the two compounds LDN193189 and SB431542 increased the three neuroectodermal mRNAs.

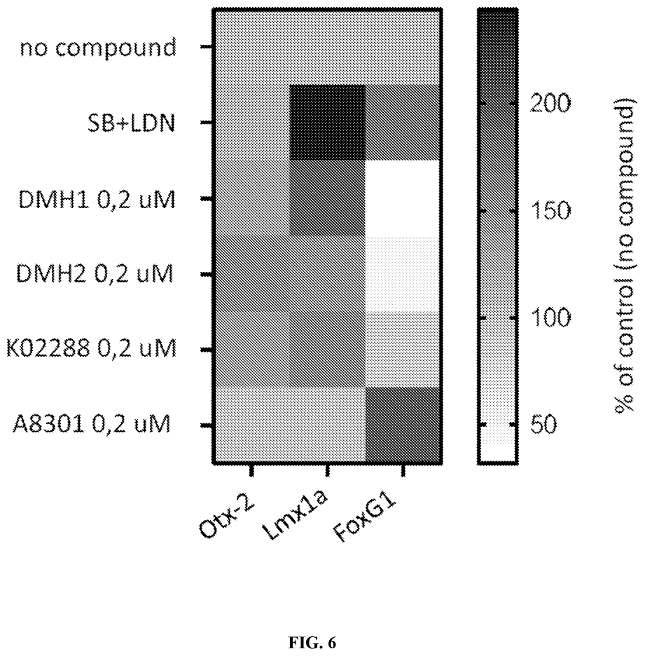

A possible impact of ALK inhibitors on early neural specification was analysed after 8 days of differentiation and compared with dual SMAD inhibition. Several markers of early specification, focusing on forebrain/midbrain regionalization were measured by Q-RT-PCR at day 8 in neural induction medium. The following markers were tested: Otx-2 which is an anterior marker (forebrain and midbrain), Lmxla which is expressed in dorsal and ventral midbrain, FoxA2 which is a ventral marker in forebrain, midbrain and hindbrain. Was also tested the forebrain FoxG1 and Msx-1 which is more specific for early dopaminergic differentiation. Several experiments were performed (n=3) and values were normalized as the percentage of control (=no compound, vehicle lone). A heat map representation is shown in FIG. 6. At this early stage (day 8), FoxA2 and Msx-1 were not detected by PCR in all conditions, showing the absence of acquisition of a ventral and dopaminergic identity. Exposition to dual SMAD inhibition (LDN193189+SB431542) show a moderate increase of Otx-2 and a more pronounced upregulation of Lmxla and FoxG1. The Hindbrain marker HoxA2 was not detected at this stage (not shown), showing the absence of a hindbrain identity (not shown). DMH1 and DMH2 at the concentration of 0.2 uM decreased the forebrain specification, as indicated by the reduced FoxG1 expression. In contrast, A8301 induced an upregulation of FoxG1, suggesting an increase of the specification towards the forebrain. Together, this early-stage analysis shows that some ALK inhibitors, in addition the neural induction similar to dual SMAD inhibition, interfere with early events of neural specification. DMH1 and DMH2 reduce the forebrain identity, a useful prerequisite for dopaminergic specification.

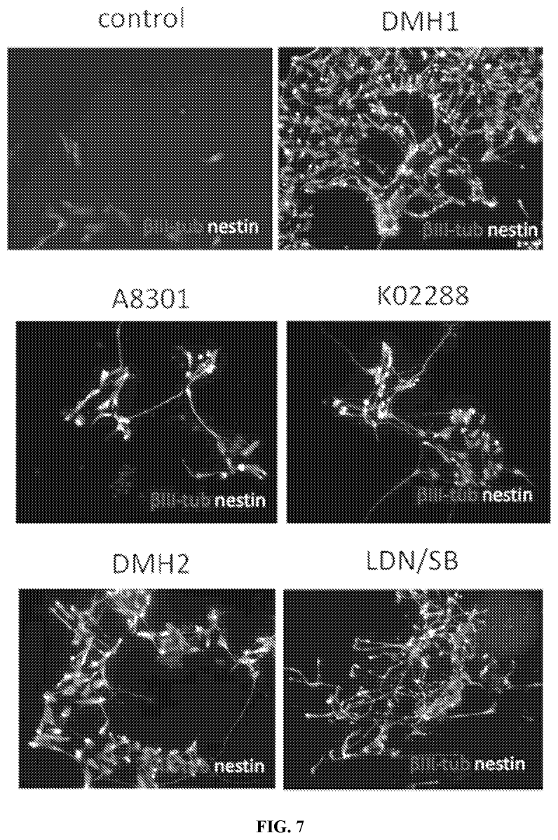

At confluency (occurring in these conditions at day 8), cells were passaged at a very low density with Accutase and polyornithine/Matrigel-coated dished for neuronal maturation in appropriated medium. Neuronal cells (nestin− and βIII-tubulin+ cells with a neuritic morphology) were present in cells previously exposed to the four compounds, in contrast to control conditions from which cells reduced progressively their number, with only few neuronal cells in the plate (FIG. 7). It confirmed that the four compounds DMH1, DMH2, K02288 and A8301, similarly to LDN193189+SB431542 condition, favoured neural induction with the generation of cells able to be further maturated into neurons.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method for differentiating stem or progenitor cells into neural cells, the method comprising contacting the cells with a compound selected from DMH1, DMH2, K02288, A8301, or combinations thereof.

2. A method for producing neural cells from stem or progenitor cells, the method comprising contacting the cells with a compound selected from DMH1, DMH2, K02288, A8301, or combinations thereof.

3. The method of claim 1 or 2, wherein the compound consists of DMH1, DMH2, K02288, or A8301.

4. The method of any one of claims 1-3, wherein the compound comprises or consists of DMH2.

5. The method of claim 1 or 2, wherein the compound consists of DMH1 and DMH2.

6. The method of claim 1 or 2, wherein the compound consists of K02288 and DMH2.

7. The method of claim 1 or 2, wherein the compound consists of A8301 and DMH2.

8. The method of any one of claims 1-7, wherein the stem or progenitor cells comprise induced pluripotent stem cells (iPSCs) or embryonic stem (ES) cells.

9. The method of claim 8, wherein the stem or progenitor cells comprise embryonic stem (ES) cells.

10. The method of claim 9, wherein the ES cells are human ES cells.

11. The method of claim 9, wherein the cells comprise HS420 cells.

12. The method of any one of claims 1-7, wherein the stem or progenitor cells comprise totipotent, pluripotent, or multipotent stem cells.

13. The method of any one of claims 1-12, wherein the contacting the cells comprises contacting the cells for a time period of about 1-7 days of substantially continuous contact.

14. The method of any one of claims 1-13, wherein the cells are contacted with 0.01-5 μM compound.

15. The method of claim 14, wherein the cells are contacted with 0.2 μM compound.

16. The method of any one of claims 1-15, wherein the cells are in serum-free medium.

17. The method of any one of claims 1-16, wherein the density of the cells in contact with the compound is 40000-60000 cells/cm².

18. The method of any one of claims 1-17, wherein the cells are cultured on a substrate comprising Matrigel and/or polyornithine.

19. The method of any one of claims 1-17, wherein the method further comprises contacting the cells with a Rho Kinase (ROCK) inhibitor.

20. The method of claim 19, wherein the ROCK inhibitor comprises Y27632.

21. The method of claim 19 or 20, wherein the cells are contacted with 5-15 μM ROCK inhibitor.

22. The method of any one of claims 19-21, wherein the density of the cells in contact with the ROCK inhibitor is 5000-15000 cells/cm².

23. The method of any one of claims 19-22, wherein the cells are cultured on a substrate comprising laminin.

24. The method of any one of claims 1-23, wherein the cells are contacted with the ROCK inhibitor prior to contact with the Compound.

25. The method of claim 24, wherein the cells are contacted with the ROCK inhibitor for a time period of 1-48 hours.

26. The method of claim 24 or 25, wherein the cells are contacted with the ROCK inhibitor

27. The method of any one of claims 1-21, wherein the method excludes contacting the cells with a Smad inhibitor and/or a BMP4 inhibitor.

28. The method of claim 27, wherein the method excludes contacting the cells with LDN193189 and/or SB431542.

29. The method of any one of claims 1-28, wherein the method excludes dual or mono-Smad inhibition.

30. The method of any one of claims 1-29, wherein the method excludes contacting the cells with a Noggin protein.

31. The method of any one of claims 1-30, wherein the neural cells are further defined as dopaminergic neurons, glutamatergic, serotoninergic, cholinergic, GABAergic, motoneurons, astrocytes, or oligodendrocytes.

32. The method of any one of claims 1-31, wherein contacting the cells with a compound comprises culturing the cells in a cell culture medium comprising the compound.

33. The method of claim 32, wherein the cell culture medium comprises one or more of DMEM medium, DMEMF12 medium, Neurobasal medium, antimicrobial agents, B-27 supplement, N-2 supplement and L-glutamine.

34. The method of any one of claims 19-33, wherein the cells are in serum-free medium.

35. The method of any one of claims 1-34, wherein the neural cells are further defined as Nestin+, Pax-6+, and Sox-1+ cells.

36. The method of any one of claims 1-35, wherein the method further comprises selecting cells that are Nestin+, Pax-6+, and/or Sox-1+.

37. A neural cell produced by the method of any one of claims 1-36.

38. A population of cells produced by the method of any one of claims 1-36.

39. The population of cells according to claim 38, wherein the percentage of non-neural cells in the cell culture after contact with the compound for a period of time is less than 30%.

40. The population of cells according to claim 39, wherein the period of time is 4-8 days.

41. A method of treating a disease in a mammalian subject comprising administering to the subject a therapeutically effective amount of the population of neural cells of claim 38.

42. The method of claim 41, wherein the disease comprises a neurodegenerative disease.

43. The method of claim 41 or 42, wherein the subject is a human subject.

44. A method of screening a test compound comprising:
(a) contacting the test compound with the cells of claim 38; and
(b) measuring the function, physiology, or viability of the cells.

45. A method for differentiating stem or progenitor cells into neural cells, the method comprising culturing the cells in medium comprising an ALK inhibitor for 1-10 days, wherein the ALK inhibitor consists of a compound selected from DMH1, DMH2, K02288, A8301; and
wherein the method excludes contacting the cells with a SMAD inhibitor or BMP4 inhibitor.

46. A method for differentiating stem or progenitor cells into neural cells, the method comprising culturing the cells in medium comprising 10 μM of ROCK inhibitor for 2-48 hours, removing the medium and culturing the cells in medium comprising 0.2 μM of an ALK inhibitor for 1-10 days, wherein the ROCK inhibitor consists of Y27632 and the ALK inhibitor consists of a compound selected from DMH1, DMH2, K02288, A8301, or combinations thereof, and wherein the method excludes contacting the cells with a SMAD inhibitor or BMP4 inhibitor.


SEQUENCE LISTING (6 sequences)

SEQ ID NO: 1 - DNA, 20 bp, artificial sequence
  synthetic polynucleotide
acatctggctccatgttggg

SEQ ID NO: 2 - DNA, 20 bp, artificial sequence
  synthetic polynucleotide
ggcagcatgcaggagtatga

SEQ ID NO: 3 - DNA, 20 bp, artificial sequence
  synthetic polynucleotide
aatactggagacgaacgccg

SEQ ID NO: 4 - DNA, 20 bp, artificial sequence
  synthetic polynucleotide
aacccaagtctggtgtcagc

SEQ ID NO: 5 - DNA, 20 bp, artificial sequence
  synthetic polynucleotide
gttgaccaacgctggcggga

SEQ ID NO: 6 - DNA, 20 bp, artificial sequence
  synthetic polynucleotide
cgggctacctcctccgtcgt